(19) United States
(12) Patent Application Publication
(10) Pub. No.: US 2004/0235965 A1
(43) Pub. Date: Nov. 25, 2004

(54) Utilization of rutins and aescins in the treatment of circulatory disturbances of the ear

(76) Inventor: Kailash Kumar Gauri, Hitzhusen (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/477,145

(22) PCT Filed: May 10, 2002
(86) PCT No.: PCT/EP02/05177

(30) Foreign Application Priority Data
May 10, 2001 (DE) 101 22 715.9

(51) Int. Cl. A61K 31/00

(52) U.S. Cl. 514/822

(57) ABSTRACT

The invention relates to the utilization of rutins and aescins in the treatment of circulatory disturbances of the ear. The invention also relates to agents with a corresponding active ingredient combination and agents in the form of commercial packagings with corresponding combination preparations or monopreparations for combined application.

The present invention relates to the use of rutins and escins for the treatment of circulatory disturbances of the ear. Also described are compositions containing a corresponding active compound combination and compositions in the form of commercial packs containing corresponding combination preparations or monopreparations for combined administration.

Rutins are glycosides of the flavone quercetin which occur in many plant species. Rutin, which is known under the international nonproprietary name (INN) rutoside, is usually employed in the form of the acidic sodium salts against capillary hemorrhages and further conditions accompanying increased capillary brittleness and membrane permeability. It was therefore often designated as an “antipermeability factor” or as vitamin P.

Instead of rutin, synthetic rutin derivatives are frequently also used. These include, in particular, O-(β-hydroxyethyl)rutins, which are often obtained as a mixture of rutin which is substituted 1 to 4 times and on different positions of the quercetin by hydroxyethyl groups. An important representative of these derivatives is troxerutin, which can be employed as the main component of a mixture of O-(β-hydroxyethyl)rutins or as a pure substance for the treatment of disorders of the veins and sequelae, in particular in chronic venous insufficiency, varicosis, varicose ulcer and thrombophlebitis. Further indication areas should be ophthamological applications, such as diabetic retinopathy, retinal and vitreous body hemorrhages, subconjunctival hemorrhages and thromboses.

In the case of escin, a saponin mixture isolable from horse chestnuts, a common venous therapeutic is concerned, which is valued as an escin-containing chestnut seed extract or as a purified asecin because of an edemoprotective or antiexudative action.

Accordingly, in the field of venous therapeutics combination preparations are also supplied which contain representatives of both classes of active compound. For instance, horse chestnut seed extracts containing O-(β-hydroxyethyl)rutins or rutin sulfuric acid esters are recommended in venous circulatory disturbances, such as edema, cramps in the calf, itching, and pain and a feeling of heaviness in the legs, swellings and congestive states caused by varicose veins, varicosis and post-thrombotic syndrome, ulcers of the leg, hemorrhoids, and post-traumatic and postoperative soft-tissue swellings (cf. Rote Liste 2000, Aulendorf: ECV, Editio Cantor Verlag, entries 83044 and 83046).

WO 98/51291 further mentions, inter alia, rutoside, troxerutin or escin for the treatment and prevention of ischemic disturbances.

Circulatory disturbances of the ear, in particular if they concern the inner ear, lead to an often irreversible loss of function of the ear, i.e. to impaired hearing or even deafness.

Circulatory disturbances of the ear are therefore symptoms that must be taken seriously and require effective treatment. If these symptoms are attributable to systemic vascular disorders, treatment is as a rule directed at the underlying disorder. However, the therapeutic success achievable in this way, aimed at the symptomatology in the ear, is often not satisfactory.

It is therefore frequently necessary in these cases to take further measures in addition to treatment of the underlying disorder.

The hearing aids generally prescribed by otologists for this purpose are however disadvantageous due to lack of patient acceptance and unselective amplification of sounds. Even systemic therapies using circulation-promoting agents, such as, for example, pentoxyphylline or pentyphylline, do not offer any satisfactory possibility of treatment for the field of circulatory disturbances of the ear.

It has now been found that certain combined uses of rutins and escins open up a surprisingly effective possibility of treatment of circulatory disturbances of the ear.

The present invention therefore relates to the use of at least one flavonoid from the group consisting of the rutins, namely of rutin, physiologically acceptable derivatives and/or salts thereof, in combination with at least one saponin from the group consisting of the escins, namely of escin, physiologically acceptable derivatives and/or salts thereof, for the treatment of circulatory disturbances of the ear.

The use according to the invention of rutin, physiologically acceptable derivatives or salts thereof—for the purpose of simplification also designated as “rutins” or “rutin component”—and the use of escin, physiologically acceptable derivatives or salts thereof—for the purpose of simplification also designated as “escins” or “escin component”—offers significant advantages in the treatment of circulatory disturbances of the ear.

The rutin component and the escin component can in principle be administered together in one formulation or separately in at least two different formulations. The latter possibility comprises both the simultaneous and the temporally separate administration, i.e. taking place at different points in time. A particular embodiment of the temporally separate administration is realized by the alternate administration of both components, for example with an early/late diurnal rhythm.

In this sense, the invention relates to compositions for the treatment of circulatory disorders of the ear, which are based on a combination of at least one rutin, of a physiologically acceptable derivative and/or salt thereof and at least one escin, of a physiologically acceptable derivative and/or salt thereof, and, if appropriate, further active compounds, where the active compound components, in particular the rutin component and escin component, can be formulated together or separately.

“Rutin” designates according to the invention 3-[[6-O-(6-deoxy-α-L-mannopyranosyl)-β-D-glucopyranosyl]oxy]-2-(3,4-dihydroxyphenyl)-5,7-dihydroxy-4H-1-benzopyran-4-one, also called quercetin-3-rutinoside or rutoside (INN), of the formula I

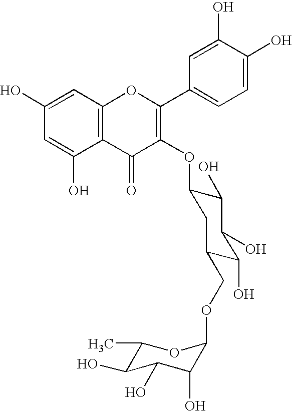

The rutin derivatives especially include O-(β-hydroxyethyl)-rutins, in particular the corresponding mono-, bis-, tris- and tetra(hydroxyethyl) derivatives including the respective regio-isomeric forms. For example, monoxerutin, i.e. 7-mono-O-(β-hydroxyethyl)rutin and especially troxerutin, i.e. 3′,4′,7-tris-O-(β-hydroxyethyl)rutin of the formula II

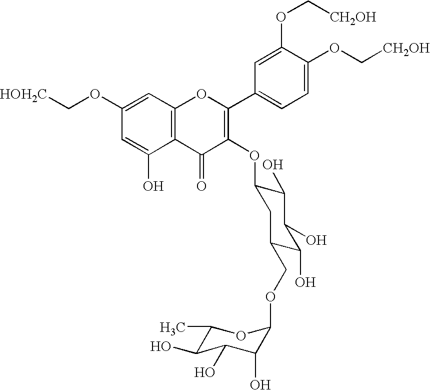

may be mentioned.

Further physiologically acceptable derivatives of rutin include, for example, ethoxazorutin, 8,8′-methylenebis[6-diethylamino-methylrutin], rutin sulfuric acid ester, diosmin (2,3-dehydro-hesperidin).

The physiologically acceptable salts of rutin or rutin derivatives in the present case preferably include base addition salts, which are formed in particular with acidic esters, e.g. the sulfuric acid esters, of rutin.

The base addition salts include salts with inorganic bases, for example metal hydroxides or carbonates of alkali metals, alkaline earth metals or transition metals, or with organic bases, for example ammonia or basic amino acids, such as arginine and lysine, amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetraamine, saturated cyclic amines having 4 to 6 ring carbon atoms, such as piperidine, piperazine, pyrrolidine and morpholine, and further organic bases, for example N-methylglucamine, creatine and tromethamine, as well as quaternary ammonium compounds, such as tetramethylammonium and the like.

Salts with inorganic bases, e.g. Na, K, Mg, Ca, Zn, Cr and Fe salts, are preferred.

Rutin can be obtained from natural sources, in particular the flower buds of Sophora japonica or buckwheat herbage. For example, the drug material can firstly be extracted with hot water or lower alcohols, the extracts obtained concentrated and optionally defatted using suitable solvents. The crude rutin depositing on cooling can then be recrystallized from water or ethanol or dissolved by addition of alkali and precipitated again using acids.

Moreover, rutin is also accessible synthetically, for example by reacting 7,4′-dibenzylquercetin with hexaacetobromorutinose under suitable conditions, for example coupling in pyridine in the presence of Ag₂CO₃. The acetyl ester groups can then be hydrolyzed and the benzyl protective groups removed, for example hydrogenolytically by means of Pd/C. If necessary, the crude rutin is recrystallized, for example from methanol.

O-(β-Hydroxyethyl)rutins can be obtained by hydroxyethylation of the phenolic groups of rutin with suitable reagents such as 2-chloroethanol or glycochlorohydrin. As a rule, this reaction is carried out in an alkaline medium, for example in the presence of NaOH.

The term “escin” describes a saponin mixture of mainly diacetylated tetra- and pentahydroxy-β-amyrin compounds isolable from horse chestnuts (Aesculus hippocastanum) and in particular from their seeds, which in position 3 carry a glucuronic acid substituted by sugar radicals, for example glucose, galactose and/or xylose. The aglycones are known under the designation barringtogenol of the formula III

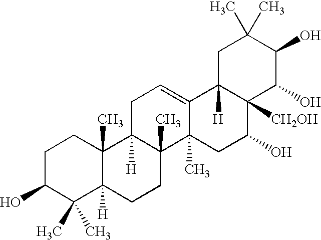

and protoescigenin of the formula IV

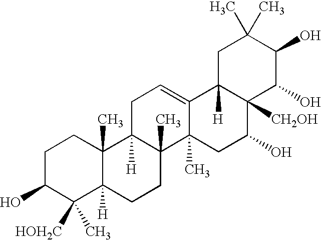

In position 21, different amounts of angelic, tiglic, α-methyl-butyric and isobutyric acid are bound in ester-like manner. The term “escin” includes α-escin, β-escin and crypto-escin, which carry acetyl groups in different positions, for example on the 22-α-hydroxyl (β-escin) or on the 28-hydroxyl (cryptoescin). Preferably, the escins are used as a horse chestnut seed extract or in isolated form.

Suitable horse chestnut seed extracts include fluid extracts which are obtainable using alcohol-water mixtures, and dry extracts which can be obtained from the fluid extracts by subsequent drying, preferably spray drying. Suitable extracting agents are, for example, aqueous ethanol or methanol. Good escin yields are obtained, for example, by extraction with 40 to 60% strength ethanol or methanol. In particular, dry extracts (4-8:1) which are standardized on triterpene glycosides, calculated as escin, are common.

Isolated escin can be isolated from horse chestnut seeds, for example, by means of chromatography using ion exchangers (resins).

For further explanation, reference may be made, for example, to EP 0 900 563 A1, which relates to the preparation of escin-containing pharmaceutical preparations.

The physiologically acceptable derivatives of escin in the present case include, for example, esters with preferably organic acids, in particular carboxylic acids, e.g. acetic acid, tartaric acid, lactic acid, citric acid, malic acid, mandelic acid, ascorbic acid, maleic acid, fumaric acid, gluconic acid or sulfonic acids, e.g. methanesulfonic acid, benzenesulfonic acid and toluenesulfonic acid, and the like. Acids of this type are mainly bonded to one or more OH groups in position 21, 22 and also 28. The tartrate, for example, has proven expedient.

In addition to the rutin and escin components, the treatment according to the invention can additionally include further active compounds. These active compounds can be, in particular, those whose action is similar to the rutin- or escin-mediated action or supplements this. Thus it can be advantageous, additionally to the combination according to the invention, to administer otologicals, venous therapeutics, anti-hemorrhagics, anticoagulants and similar active compounds. In particular, it can be expedient to administer antiinflammatory active compounds of the corticoid type, e.g. glucocorticoids, or of the noncorticoid type, such as, for example, indomethacin, or acetylsalicylic acid or derivatives thereof. Likewise, it can be expedient to administer thrombolytic active compounds, such as, for example, streptokinase or urokinase, and anticoagulants, such as, for example, coumarin derivatives.

A particular embodiment of the present invention is based on the combination of troxerutin with escin or a physiologically acceptable salt thereof.

Circulatory disturbances of the ear are understood as meaning circulatory disturbances which affect the ear or parts thereof or impair its/their functioning. These especially include circulatory disturbances of the inner ear, in particular in the region of the vascular stria, the cochlea, the auditory nerve, the spiral ganglion or the auditory pathway, and also circulatory disturbances of the auditory center in the brain, in particular of the temporal lobe of the brain. According to a particular embodiment, the present invention therefore relates to the acute or preventive treatment of circulatory disturbances of the inner ear and of the auditory pathway. Occasionally, the circulatory disturbances treatable according to the invention also include those disturbances which can lead to circulatory disturbances of the ear without such a condition having to be present at the time of treatment. These include, for example, circulatory disturbances in the vertebrobasilar region, chronic circulatory disturbances associated with disturbances of blood pressure regulation, or, for example, generalized arteriosclerosis or angiopathy due to a metabolic disorder such as e.g. diabetes mellitus. A treatment of such disturbances represents a prevention of circulatory disturbances of the ear.

Disturbances treatable according to the invention which originate from circulatory disturbances of the ear especially include disorders which are attributable to venous insufficiency, in particular to thrombotic changes of the branch veins or central veins supplying the ears, and/or arterial insufficiency, e.g. teleangiectases. These disturbances are especially hearing impairment and in particular presbyacusis, and also tinnitus and sudden deafness of vascular origin (both unilateral and bilateral) as well as the restrictions of hearing associated therewith. Vestibular symptoms, in particular disturbances of balance and/or vertigo, are also included here.

Preferred embodiments of the present invention are aimed at the treatment of cochlear hearing disorders, in particular those of vascular origin, which include presbyacusis, hypacusis, dysacusis, sudden deafness, tinnitus, sensorineural hearing loss and also vestibular symptoms such as balance disturbance and/or vertigo.

The use according to the invention gains in importance in adults with increasing age. In the group consisting of the over 40-year olds and especially the over 50-year olds, the treatment is accompanied by particular advantages. Patients suffering from a cochlear hearing impairment of vascular origin, which manifests itself as sudden deafness or balance disturbance, form a further group in which the treatment according to the invention can be accompanied by particular advantages.

Disorders to be treated according to the invention are frequently characterized by progressive development, i.e. the conditions described above change in the course of time, as a rule the degree of severity increases and conditions may change into one another or conditions further to already existing conditions can occur. Preventive therapy can in particular be important if changes are detected in the region of the small vessels (microangiopathies) of organs or body parts other than the ears.

A particular aspect of a treatment within the meaning according to the invention relates to the treatment of acute or chronic disturbances. The treatment can be accomplished symptomatically, for example as symptom suppression. It can be carried out short-term, be accomplished medium-term, or it can also be a long-term treatment, for example in the course of a maintenance therapy.

According to the invention, an efficacious amount of rutin component and an efficacious amount of escin component, as a rule formulated corresponding to pharmaceutical, veterinary pharmaceutical or foodstuffs technological practice, is administered to the individual to be treated, preferably a mammal, in particular a human and also an agricultural animal or domestic pet.

The treatment is as a rule carried out by single or repeated daily administration of a suitable dose optionally together or alternatively with other active compounds or active compound-containing preparations, so that an individual to be treated of approximately 75 kg body weight is administered a daily dose of approximately 10 mg to 20 g, preferably of approximately 200 mg to 10 g, advantageously of approximately 900 mg to 5 g and in particular of approximately 2 g to 3 g of rutin component, and of approximately 500 μg to 1 g, preferably of approximately 1 mg to 500 mg and in particular of approximately 5 mg to 200 mg of escin component, on oral administration, and of approximately 10 mg to 20 g of rutin component of approximately 25 μg to 500 mg of escin component on parenteral or alternatively intraauricular administration. Independently of the escin dose, the administration of an oral daily dose of more than 1 g, preferably of more than 1.8 g and in particular of more than 2 g of rutin component represents a particularly advantageous aspect of the invention.

Amounts and proportions of active compounds relate to the active compound, so that for salts and derivatives an appropriate conversion has to be carried out. An adaptation to the body weight may be necessary.

The invention also relates to the production of compositions for the treatment of an individual, preferably of a mammal, in particular of a human and also of an agricultural animal or domestic pet.

One aspect of the present invention is therefore also compositions comprising

i) at least one flavonoid from the group consisting of the rutins, namely rutin, physiologically acceptable derivatives and/or salts thereof, and

ii) at least one saponin from the group consisting of the escins, namely escin, physiologically acceptable derivatives and/or salts thereof, and

optionally at least one further active compound and a formulation base.

Compositions according to the invention are therefore based on an active compound combination and optionally a formulation base.

The compositions in particular include pharmaceutical compositions, by which veterinary medicinal compositions are also intended. Otological compositions represent one particular embodiment.

The active compound combination within the meaning of the invention comprises, as active compound component i), at least one rutin, i.e. rutin, physiologically acceptable derivatives and/or salts thereof. Mixtures of these forms are possible and to be taken into consideration in certain cases. According to a particular embodiment, the active compound component i) consists of an O-(β-hydroxyethyl)rutin mixture, which contains troxerutin as the main component, preferably to at least 50% by weight and in particular to at least 80% by weight. According to a further particular embodiment, the active compound component i) consists essentially of troxerutin. The percentage by weight details are based on the total weight of the active compound component i).

The active compound combination within the meaning of the invention comprises, as active compound component ii), at least one escin, i.e. escin, physiologically acceptable derivatives and/or salts thereof. Mixtures of these forms are possible and to be taken into consideration in certain cases. According to a particular embodiment, the active compound component ii) consists of a horse chestnut seed extract, which preferably contains approximately 10 to 30% by weight of escin. According to a further particular embodiment, the active compound component ii) consists essentially of escin. The percentage by weight details are based on the total weight of the active compound component ii).

Furthermore, the active compound combination within the meaning of the invention can comprise as active compound component iii) further active compounds, for example the active compounds mentioned above in this connection.

The proportion of the active compound combination in the formulation is greater than a proportion optionally present in natural sources. In this sense, the compositions according to the invention are enriched with respect to the active compound combination. In the case of a pharmaceutical composition, the proportion is as a rule of approximately 1 to 60% by weight, preferably approximately 5 to 35% by weight and in particular approximately 10 to 30% by weight.

Details in % by weight relate, if not stated otherwise, to the total weight of the formulation.

The formulation base of formulations according to the invention contains physiologically acceptable auxiliaries. Physiologically acceptable auxiliaries are those which it is known can be used in the field of pharmacy, foodstuffs technology and related fields, in particular those listed in relevant pharmacopeia (e.g. DAB, Ph. Eur., BP, NF), and also other auxiliaries whose properties do not stand in the way of a physiological application.

Suitable auxiliaries can be: wetting agents; emulsifiers and suspending agents; preservatives; antioxidants; antiirritants; chelating agents; pan-coating auxiliaries; emulsion stabilizers; film-forming agents; gel-forming agents; odor-masking agents; taste corrigents; resins; hydrocolloids; solvents; solubilizers; neutralizing agents; permeation accelerators; pigments; quaternary ammonium compounds; refatting and superfatting agents; ointment, cream or oil bases; silicone derivatives; spreading aids; stabilizers; sterilizing agents; suppository bases; tablet auxiliaries, such as binders, fillers, lubricants, disintegrants or coatings; propellants; drying agents; opacifiers; thickeners; waxes; plasticizers; white oils. A relevant embodiment is based on expert knowledge, as is shown, for example, in Fiedler, H. P., Lexikon der Hilfsstoffe für Pharmazie, Kosmetik and angrenzende Gebiete [Encyclopedia of excipients for pharmacy, cosmetics and related fields], 4th edition, Aulendorf: ECV-Editio-Kantor-Verlag, 1996.

The sum of active compound component and formulation base is as a rule 100% by weight.

Examples of suitable pharmaceutical formulations are solid pharmaceutical forms, such as powders, granules, tablets, in particular film-coated tablets, pastilles, sachets, cachets, sugar-coated tablets, capsules such as hard and soft gelatin capsules, suppositories or vaginal pharmaceutical forms, semisolid pharmaceutical forms, such as ointments, creams, hydrogels, pastes or patches, and liquid pharmaceutical forms, such as solutions, emulsions, in particular oil-in-water emulsions, suspensions, for example lotions, injection and infusion preparations, eardrops. Implanted delivery devices can also be used for the administration of active compounds according to the invention. Further, liposomes or microspheres can also be used. Solid pharmaceutical forms and in particular capsules or tablets are preferred.

The formulations can be administered, for example, by the oral, rectal, transdermal, subcutaneous, intravenous, intramuscular, intraauricular or intranasal route. Oral administration is preferred.

In the preparation of the compositions, the active compounds are usually mixed or diluted with a suitable auxiliary, in this case also to be designated as an excipient. Excipients can be solid, semisolid or liquid materials, which serve as a vehicle, carrier or medium for the active compound. The admixture of further auxiliaries is carried out if necessary in a manner known per se. Shaping steps, if appropriate in combination with mixing processes, can be carried out, e.g. granulation, compression and the like.

In particular, the active compound components can be formulated together. They can, however, also be separately processed first and then combined in a compartmentalized, e.g. multilayer pharmaceutical form. By this means, possible active compound incompatibilities and different active compound properties, such as bioavailability, stability, solubility and the like, can be taken into account.

The invention likewise relates to corresponding monopreparations in the form of commercial packs, from which the combined use according to the invention is to be inferred.

The present invention is illustrated in greater detail with the aid of the following examples, without being restricted thereto.

### EXAMPLE 1

Pharmaceutical compositions

| a) Soft gelatin capsules containing troxerutin and escin | | |
|---|---|---|
| (troxerutin 450 mg + escin 25 mg) | | |
| Filling: | | |
| troxerutin | 450 | mg |
| eschin | 25 | mg |
| soybean oil (refined) | 440 | mg |
| soybean lecithin (E322) | 50 | mg |
| highly disperse silica | 5 | mg |
| capsule shell: | | |
| gelatine | 303 | mg |
| glycerol 85% | 87 | mg |
| sorbitol 70% | 77 | mg |
| purified water | 52 | mg |
| iron oxide pigment Brown 75 ( E 172) | 3 | mg |
| b) Tablet containing troxerutin and escin | | |
| (troxerutin 250 mg + escin 13.75 mg) | | |
| troxerutin | 250 | mg |
| escin | 13.75 | mg |
| lactose | 127.5 | mg |
| magnesium stearate | 5 | mg |
| talc | 23.75 | mg |
| microcrystalline cellulose | 81 | mg |
| c) Hard gelatin capsule containing troxerutin and escin | | |
| (troxerutin 600 mg + escin 33 mg) | | |
| Filling: | | |
| troxerutin | 600 | mg |
| escin | 33 | mg |
| lactic acid | 272.5 | mg |
| magnesium stearate | 14.5 | mg |
| talc | 30 | mg |
| alginic acid | 50 | mg |

d) Furthermore, tablets or sugar-coated cores prepared according to c) can
be provided in a known manner with a film coating which is soluble in the
stomach or in the small intestine.

### EXAMPLE 2

Efficacy

### CASE EXAMPLE 1

71-year-old male patient with hearing impairment.

Sensorineural hearing loss existed which was confirmed by pure-tone threshold audiometry to be a loss of hearing especially in the high frequency range. These findings existed for 8 months with the tendency to worsen. The underlying disease present was a generalized sclerotic vascular condition; the medical history further indicated an acoustic trauma.

A 4-week therapy with 125 mg of escin orally daily was first carried out. The findings did not improve under this. No change was detecable by pure-tone threshold audiometry.

After a three-week therapy break, a 4-week therapy with 2250 mg of troxerutin orally daily followed. Under this, an improvement in the high-frequency range by 10 dB was observed in the audiogram for the right ear.

After a 3-week therapy break, findings once again deteriorated. Therapy with the combination of 2250 mg of troxerutin and 125 mg of escin daily orally was carried out. After 1 week the audiogram again improved by 10 dB, after 2 weeks by 20 dB (measured from baseline prior to the start of therapy). The findings subsequently remained unchanged until the end of therapy.

### CASE EXAMPLE 2

67-year-old female patient with slowly progressive hearing impairment, which had existed for 5 months.

Sensorineural hearing loss existed which was confirmed by pure-tone threshold audiometry to be a loss of hearing both in the low frequency and the high frequency range. The underlying diseases present were a sclerotic vascular condition and latent diabetes mellitus.

A 4-week therapy with 125 mg of escin orally daily was carried out. The findings did not improve as a result of this.

After a 3-week break, a three-week therapy with 2250 mg of troxerutin orally daily was carried out. After 1 week, the audiogram improved by 10 dB in the low frequency range, along with a minor improvement in the high frequency range. The course subsequently remained stationary.

After a 3-week therapy break, in which deterioration by 10 dB reoccurred in the low frequency range, a 4-week therapy with 2250 mg of troxerutin and 125 mg of escin orally followed. After 1 week, there was an improvement by 15 dB and by 10 dB, and after 3 weeks, by 25 dB and by 20 dB, in the low and high frequency ranges, respectively. Findings subsequently remained stable until the end of therapy.

### CASE EXAMPLE 3

32-year-old female patient with tinnitus and vertigo attacks for 4 months.

Moderate hearing loss existed which was confirmed by pure-tone threshold audiometry to be a loss of hearing especially in the low and high frequency ranges. The underlying disease present was insulin-dependent diabetes mellitus. The findings existed for 4 months with the tendency for progressive worsening.

A 4-week therapy with 2250 mg of troxerutin orally daily followed. Under this, hearing improved in the 1st week by 10 dB in the low frequency range and by 5 dB in the high frequency range. There was no change in the frequency of vertigo attacks (1 to 4 times within 2 days). In the 4th week of treatment, the hitherto incessant tinnitus became intermittent with relatively frequent absences which lasted for hours.

A 3-week therapy break followed. Hearing again deteriorated by 5 dB in both the low and high frequency ranges toward the end of this therapy break. The tinnitus reverted to its previous pattern.

Therapy with 125 mg of escin orally daily now began. No improvement occurred, the findings deteriorated by a further 5 dB in the low frequency range. There was no change with regard to vertigo attacks or tinnitus.

After a 3-week therapy break, therapy with the combination of 2250 mg of troxerutin and 125 mg of escin orally daily was carried out over 3 weeks. After one week, hearing improved by 15 dB and by 10 dB, and after 3 weeks, by 25 dB and by 15 dB, in the low and high frequency ranges, respectively. Vertigo attacks occurred less frequently (twice within one week) and ceased in the 4th week of treatment. From week 2, tinnitus was intermittent with absences first lasting for some hours and then in week 4 for about half the day, and was totally absent on one day.

### CASE EXAMPLE 4

36-year-old male patient with stationary hearing impairment for 4 months.

Sudden deafness existed which was confirmed by pure-tone threshold audiometry to be associated with loss of hearing both in the low frequency and the high frequency range. The medical history indicated an acoustic trauma. No other risk factors existed.

A 4-week therapy with 125 mg of escin orally daily followed. Findings remained unchanged.

After a 3-week therapy break, therapy with 2250 mg of troxerutin orally daily followed. In week 3, hearing improved by 10 dB in the low frequency range and marginally in the high frequency range.

A 3-week therapy break followed in the course of which the loss of hearing increased by 10 dB in the low frequency range.

A therapy with 2250 mg of troxerutin and 125 mg of escin orally daily now followed. After 2 weeks, hearing improved by 10 dB and by 5 dB, and after 4 weeks by 20 dB and by 10 dB, in the low and high frequency ranges, respectively.

1-15. (Cancelled).

16: A method for the treatment of circulatory disturbances of the ear, which comprises administering at least one rutin in combination with at least one escin to a subject in need thereof.

17: The method as claimed in claim 16, wherein the rutin and the escin are administered together or separately.

18: The method as claimed in claim 16, wherein the rutin is an O-(β-hydroxyethyl) rutin.

19: The method as claimed in claim 18, wherein the O-(β-hydroxyethyl) rutin is troxerutin.

20: The method as claimed in claim 16, wherein the escin is present in the form of a horse-chestnut seed extract.

21: The method as claimed in claim 16, wherein the circulatory disturbances of the ear are circulatory disturbances of the inner ear and of the auditory pathway.

22: The method as claimed in claim 21, wherein the circulatory disturbances of the inner ear and of the auditory pathway are microcirculatory disturbances of the inner ear and the auditory center in the brain.

23: The method as claimed in claim 22, wherein the microcirculatory disturbances affect the vascular stria, the cochlea, the auditory nerve, the spiral ganglion, the auditory pathway or the temporal lobe of the brain.

24: The method as claimed in claim 16, wherein the circulatory disturbances of the ear are hearing impairment.

25: The method as claimed in claim 24, wherein the hearing impairment is presbyacusis.

26: The method as claimed in claim 16, wherein the circulatory disturbances of the ear are disturbances of balance and/or vertigo.

27: The method as claimed in claim 16, wherein the circulatory disturbances of the ear are noises in the ear.

28: The method as claimed in claim 16, wherein the circulatory disturbances of the ear are sudden deafness of vascular origin.

29: The method as claimed in claim 16, where a daily dose of more than 1800 mg of at least one rutin and of approximately 1 mg to 500 mg of at least one escin are administered orally to the subject or bioequivalent amounts thereof are administered in another way.

30: A method for the treatment of circulatory disturbances of the inner ear and of the auditory pathway, which comprises administering troxerutin in combination with escin to a subject in need thereof.

31: The method as claimed in claim 30, wherein troxerutin is adminstered orally at a daily dose of approximately 900 mg to 5000 mg and escin is adminstered orally at a daily dose of approximately 5 mg to 200 mg.

32: The method as claimed in claim 31, wherein the daily dose of troxerutin is more than 2000 mg.
